(19) United States
(12) Patent Application Publication
(10) Pub. No.: US 2025/0345491 A1
(43) Pub. Date: Nov. 13, 2025

(54) IMPROVED NERVE REGENERATION SCAFFOLD FOR ACCELERATED REGROWTH

(71) Applicants: LINTEC OF AMERICA, INC., Plano, TX (US); HIROSHIMA UNIVERSITY, Hiroshima (JP)

(72) Inventors: Akira KODAMA, Hiroshima (JP); Masakazu ISHIKAWA, Hiroshima (JP); Takahiro UEDA, Kusatsu City (JP); Marcio D. LIMA, Richardson, TX (US)

(73) Assignees: LINTEC OF AMERICA, INC., Plano, TX (US); HIROSHIMA UNIVERSITY, Hiroshima (JP)

(21) Appl. No.: 18/994,865

(22) PCT Filed: Aug. 25, 2023
(86) PCT No.: PCT/US2023/031153

§ 371 Date: Jan. 15, 2025

Related U.S. Application Data
(60) Provisional application No. 63/402,177, filed on Aug. 30, 2022.

(51) Int. Cl. A61L 29/02; A61F 2/02; A61L 29/04; A61L 29/14

(52) U.S. Cl. CPC A61L 29/02; A61F 2/02; A61L 29/045; A61L 29/046; A61L 29/148; A61F 2210/0004; A61L 2400/12; A61L 2430/32

(57) ABSTRACT

Improved nerve regeneration scaffolds are disclosed, which include a plurality of modified nanotube yarn bundles disposed of within the scaffold lumen. The modified nanotube yarn bundles have enhanced hydrophilicity and water absorption. They are separated by distances to form channels corresponding to nerve fiber diameters to be occupied by regenerative nerve tissues. The channel walls have gaps between the yarn bundles for enhanced permeability. The scaffolds have reduced inflammatory infiltration and rejection response and support individual nerve fiber regrowth with a reduced likelihood of undesirable outcomes, such as nerve pain or reduced nerve function.

### CROSS-REFERENCE TO RELATED APPLICATION

This application claims the benefit of U.S. Provisional Patent Application No. 63/402,177, filed Aug. 30, 2022, which is hereby incorporated by reference in its entirety.

### TECHNICAL FIELD

The present disclosure generally relates to improved devices with modified nanofiber yarns. Specifically, the present disclosure is related to implantable medical devices based on functionalized nanofibers or nanotube yarns and yarn bundles for nerve tissue regeneration in regenerative medicine. It focuses on peripheral nerve tissue regeneration for long-gap injuries with an oxidized nanofiber yarn bundle-based scaffold. Additional applications include neuronal cell expansion and differentiation ex vivo.

### BACKGROUND

There has been tremendous interest in neural cell culture, cell expansion, and tissue regeneration. However, the general challenges remain the same as the mature neurons do not undergo cell division easily compared to other non-neuronal cell and tissue types. Expansion of primary cells obtained or dissociated from harvested tissues from the central nervous system (CNS) and peripheral nervous system (PNS) is even more difficult. Methods and devices for securing and maintaining effective cell cultures become important for neurological studies and neurotoxicological testing.

Injury to the brain, spinal cord, or peripheral nerve tissue, especially the long-gap nerve tissue loss due to severe tissue laceration and significant debridement, often leads to either partial or complete functional loss. The highly desirable full functional recovery may be hard to achieve while complications or side effects inevitably emerge under certain circumstances.

Unlike other tissue types, merely suturing the severed ends of the nerves together is often inadequate for promoting complete restoration and healing of the damaged peripheral nerve tissues. In particular, upon severing a section of nerve, regeneration of nerve tissue to restore neural functionality may be limited, leading to most often little recovery or partial recovery of the lost nerve functions. Recovery may also be accompanied by persistent nerve pain (commonly referred to as neuropathy or neuropathic pain), among other problems. With long-gap injuries, bringing the proximal and distal ends of severed nerve to close vicinities may not be an option. By doing so, over-stretching the nerves could cause pinches and induce pain with increased tension. Many attempts and experimental techniques have been developed to improve nerve injury recovery. Among these, bridging the severed ends of nerve fibers with a conduit or a multi-channel conduit allows cells, such as Schwann cells and pluripotent stem cells, to proliferate and differentiate to form connections between the disconnected nerve ends. The conduit also deters undesirable cells from the surrounding tissues, such as fibroblast, which often have faster replication rates to preoccupy the vacant space, limiting the highly desirable nerve tissue regeneration.

Previously carbon nanotube yarns have been attempted to serve as structural and supporting material for nerve regeneration scaffolds. Carbon nanotubes are generally hydrophobic, rendering themselves less ideal for cells and tissues having amphipathic bilayers.

### SUMMARY

According to an aspect of the present disclosure, the following non-limiting exemplary embodiments or examples are provided.

Example 1 is a nerve regeneration scaffold comprising: a tube having a first end and an opposite second end, the tube defining a lumen with a first diameter and a central axis, the tube comprising a biocompatible material, and a plurality of nanofiber yarns within the lumen.

Example 2 includes the subject matter of Example 1, wherein the nanofiber yarns are modified.

Example 3 includes the subject matter of Example 2, wherein the modification is a strong oxidation process.

Example 4 includes the subject matter of Example 3, wherein the oxidation process involves an oxidation gas or an oxidation solution.

Example 5 includes the subject matter of any of Examples 2-4, wherein the modified nanofiber yarns have enhanced wettability and/or hydrophilicity compared to their wettability and/or hydrophilicity prior to the modification.

Example 6 includes the subject matter of any of Examples 2-5, wherein the modified nanofiber yarns elicit a weak immune response compared to the unmodified nanofiber yarns.

Example 7 includes the subject matter of any of Examples 1-6, wherein the nanofiber yarns are nanotube yarns or nanotube yarn bundles.

Example 8 includes the subject matter of any of Examples 7, wherein the nanotube yarns or nanotube yarn bundles comprise carbon nanotube yarns or boron nanotube yarns.

Example 9 includes the subject matter of Example 1, wherein the nanofiber yarns form channels inside the tube, within the lumen, and gaps within channel walls and between two adjacent nanofiber yarns to guide nerve tissue regeneration in a direction parallel to the central axis of the tube and fill each channel and gap partially or completely.

Example 10 includes the subject matter of Example 1, wherein the tube comprises a bio-absorbable material.

Example 11 includes the subject matter of Example 2, wherein the modified nanofiber yarns have improved biocompatibility compared to unmodified nanofiber yarns.

Example 12 includes the subject matter of Example 1, wherein the nerve regeneration scaffold further comprises at least one foreign body-type multinucleated giant cell inhibitor.

### BRIEF DESCRIPTION OF THE DRAWINGS

The present disclosure is further described in the detailed description which follows, in reference to the noted plurality of drawings, by way of non-limiting examples of preferred embodiments of the present disclosure, in which like characters represent like elements throughout the several views of the drawings.

FIG. 1 is a schematic illustration of a nerve regeneration scaffold connected with a served nerve fiber in accordance with an exemplary embodiment.

FIG. 2A is a schematic illustration of a nerve regeneration scaffold with nanofibers inside a scaffold lumen, as shown in FIG. 1, in accordance with an exemplary embodiment.

FIG. 2B is a schematic illustration of a cross-sectional view of a portion of a nerve regeneration scaffold in accordance with an exemplary embodiment.

FIG. 3 illustrates sciatic functional index (SFI) measurements obtained at predetermined intervals in accordance with an exemplary embodiment.

FIG. 4 illustrates immunohistological staining of macrophage lineage marker in tissue in accordance with an exemplary embodiment.

The figures depict embodiments of the present disclosure for purposes of illustration only. Numerous variations, configurations, and other embodiments will be apparent from the following detailed discussion. Furthermore, as will be appreciated, the figures are not necessarily drawn to scale or intended to limit the described embodiments to the specific configurations shown. For instance, while some figures generally indicate straight lines, right angles, and smooth surfaces, an actual implementation of the disclosed techniques may have less than perfect straight lines and right angles, and some features may have surface topography or otherwise be non-smooth, given real-world limitations of fabrication processes. In short, the figures are provided merely to show example structures.

### DETAILED DESCRIPTION

Through one or more of its various aspects, embodiments and/or specific features or sub-components of the present disclosure, are intended to bring out one or more of the advantages as specifically described above and noted below.

### Overview

Neurotmesis is a most severe type of nerve injury in which the nerve and its sheath are disrupted due to transactions of the nerve fibers and all supporting tissues. It creates the proximal and distal stumps of the nerve and a missing gap, which may be as short as less than one millimeter or as long as a few centimeters or longer. Such injury causes sensational abnormality due to the disruption of sensory nerve fibers and paralysis and weight reduction of the muscles because of the loss of the corresponding innervation. And the injured nerve generally does not recover without medical intervention. Symptoms of neurotmesis can be treated with opioids and anti-inflammatory drugs, while restoration of the disrupted nerve tissue and its associated function may be assisted through surgical intervention. Common surgical approaches to facilitate recovery of nerve injuries include sewing the separated nerve endings together or implanting an autologous or allographic nerve graft. Various implantable and engineered “nerve regeneration scaffolds” are still under development.

Connecting the injured ends of the nerve together by sewing may have drawbacks. For example, suturing the wounded nerve ends together may introduce unnecessary and increased tension within the treated nerve due to the missing part of nerve tissue. This tension may inhibit the regrowth of the damaged nerve and increase the possibility of scarring, making nerve function restoration inadequate. The alternative nerve graft therapy may also have drawbacks. For example, incision and removal of a portion of donor nerve tissue may cause sensory loss, function impairment, and neuropathic pain.

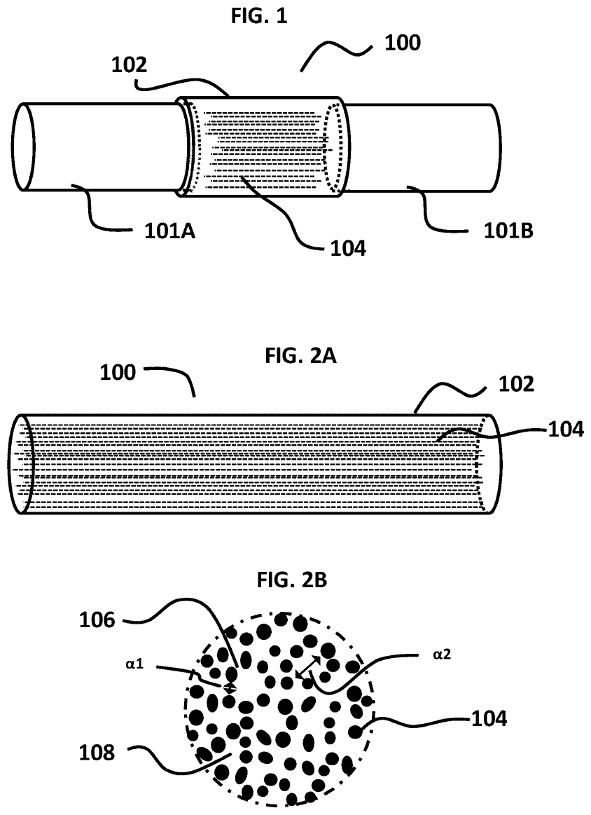

The use of a hollow nerve regeneration scaffold to facilitate the regrowth of a severed nerve is a subject of continuing research. A scaffold is a biocompatible tube sutured to both ends of the injured nerve (the stumps). In some examples, a scaffold has multiple small channels or pathways that act as a guide for the regrowing nerve fibers, while in other examples of the present disclosure, a scaffold is merely a hollow tube defining a single interior chamber. Regardless of the interior configuration, the goal of a scaffold is to create an environment facilitating the regrowth of the nerve fibers to aid the recovery of sensory function and muscle function. An exemplary nerve regeneration scaffold is schematically illustrated in FIG. 1 and FIGS. 2A-2B.

### Nerve Regeneration Scaffold

The nerve regeneration scaffold 100 in FIG. 1 has an outer housing 102 in a tubular shape, an interior space or lumen, and bundles of nanofiber yarns 104 arranged to form pathways or tunnels as a supporting framework to guide nerve tissue regeneration in an axonal manner within the lumen along the length of the tube. In FIG. 1, nerve segments 101A and 101B are the results of an injury or a surgical severance of an original intact nerve; the nerve tissue segment between 101A and 101B is removed. A nerve regeneration scaffold 100 is surgically placed between the confronting ends of the severed nerve segments 101A and 101B with proper anastomosis to facilitate nerve fiber regrowth. The scaffold length is critical in maintaining appropriate tensile load between segments 101A and 101B for nerve fiber regeneration depending on the needs and situation, which may be between less than 1.0 mm to 10.0 mm, 10.00 mm to 20.00 mm, or 20 mm or longer.

In accordance with some examples described herein, the present disclosure presents nerve regeneration scaffolds that include an outer housing fabricated from a biocompatible material. The actual size of the outer housing, i.e., the outer diameter and length, also varies, depending on requirements for regeneration. The inner diameters at both ends of the outer housing may be the same or slightly different to conform to the physiological and transient pathological characteristics of the involved nerve tissues according to various anatomic locations. The outer shape of the tube, besides a generally straight tubular shape, may also change to different curvatures according to physiological needs when implanted.

The outer tubular housing, in some instances, is fabricated from a polymeric material. In some embodiments, the polymeric material is selected from, but not limited to, polyurethane, polyester, polycaprolactone (PCL), polylactic acid (PLA), polyethylene glycol (PEG), polytetrafluoroethylene (PTFE), polymethylmethacrylate (PMMass.), an ethylene-vinyl acetate copolymer (EVA), polydimethylsiloxane (PDMS), polyester polyurethane, polyether polyurethane, polysulfone (PS), polyethylene terephthalate (PET), or a combination of one or more of the foregoing.

In some other embodiments, the selected polymeric material is a biodegradable material selected from, but not limited to, PLA, PEG, or EVA.

The outer housing prevents surrounding cells, such as fibroblasts, from migrating into the lumen of the scaffold, resulting in scar formation. Without such preventative measures, the surrounding cells may overtake the lumen space due to their much faster replication rate than the neuronal regenerative cells.

Inside the scaffold lumen, a plurality of nanofiber yarns or yarn bundles may be disposed of and further separated from each other by various distances. The nanofiber yarns may form a plurality of nanofiber yarn bundles prior to their disposition within the lumen. Together, these nanofiber yarns and/or nanofiber yarn bundles form walls of a plurality of tunnels within the lumen. The sizes of the tunnels correspond to the bundle sizes of nerve fibers; subsets of nanofiber yarns or yarn bundles form individual virtual walls. Between the yarns and yarn bundles, there are inter-bundle gaps for exchanges of nutrients and macromolecules, e.g., proteins and growth factors, across the virtual walls, and diffusions of metabolites, cellular degradations, and debris.

In one of the embodiments, the proximate nanofiber yarns can partially support individual nerve fibers due to this convenient dimensional separation between nanofiber yarns. The likelihood of the nerve fibers being tensile stressed during regrowth is reduced.

In some examples of the present disclosure, the magnitude of tensile stress on the regenerated nerve fibers is lessened relative to nerve fibers regenerated using nerve scaffolds that do not include nanofiber yarns. For at least these reasons, the likelihood of undesirable outcomes of neuropathic pain or reduced nerve function is lessened when using the embodiments of the present disclosure as a nerve regeneration scaffold.

The light-weight of the nanofiber yarn material and its high flexibility also strengthen its compatibility and fitness with biological tissues and organ systems.

### Pristine Nanotubes, Nanotube Yarns, Nanotube Yarn Bundles

According to one of the embodiments of this disclosure, the nerve regeneration scaffolds comprise nanofiber yarns in their lumen.

Nanofibers are fibers in long tubular shape with fiber diameters in a nanometer range from about 1 nanometer to about 1 micrometer. They exist in nature, for example, cellulose from plants, collagen, keratin, muscle fiber, fibrinogen from mammals, and polysaccharides from many species.

Nanofibers may be synthetic, such as polymer yarns and nanotube yarns, including carbon nanotube (CNT) yarns or boron nitride nanotube (BNNT) yarns or yarn bundles.

Other synthetic nanofibers include but are not limited to conventional carbon fibers (CCFs), cup-stacked carbon nanofibers (also known as conical CNFs), platelet carbon nanofiber, and graphene fibers which has a different carbon allotrope.

Hybrid yarns or yarn bundles produced from a natural source and a synthetic routine are becoming more prevalent in artificial tissues and organs.

Nanotube yarns are produced from synthetic nanotubes, which are long, thin, and cylindrical with a high aspect ratio.

The CNTs and BNNTs may be synthesized by standard methods. The methods vary depending on a selected end product, precursor, heating source, reaction time, reaction temperature, reaction atmosphere, catalysts, and supporting substrates. The most common methods include, but are not limited to, arc-discharge, electrolysis, laser ablation, chemical vapor deposition (CVD), flame synthesis, mechano-thermal method. The well-known CVD methods include plasma-enhanced PE-CVD, aerosol-assisted CVD (AACVD), water-assisted WA-CVD, oxygen-assisted CVD, catalytic CVD, etc.

The conventional CVD method utilizes acetylene (C₂H₂), ethylene (C₂H₄), or other hydrocarbon gas as a carbon source in a reaction chamber plus a catalyst at a reaction temperature ranging from 350° C. to 1,000° C. Amorphous boron and iron catalyst are the common choices for BNNT synthesis.

By spinning nanotube suspensions or twisting a nanotube sheet or sheets drawn from nanotube forests, nanotubes form nanotube yarns. Plying nanotube yarns may form multi-plied nanotube yarns or yarn bundles. Plied nanotube yarns may have the same nanofibers or nanotubes (homogeneous) or different nanofibers or nanotubes (heterogeneous). The nanotube fibers and nanotube yarns and yarn bundles are collectively referred to as nanotube yarn bundles hereafter.

Looping a yarn to form a spool and then cutting the spool open may be another convenient way to produce linear bundles. The Van der Waals force between nanotubes benefits the formation of close nanotube bundles.

### Oxidized Nanotube Yarn Bundles

The surfaces of nanotubes or nanotube yarns of both CNTs and BNNTs are hydrophobic and generally resistant to chemical alterations. They tolerate well under mild to moderate pH environments and are much less susceptible to enzymatic degradation. These featured stabilities are advantageous for in vivo applications as implants. Highly potent oxidizing agents may modify nanotube surfaces.

In accordance with one of the embodiments of the disclosure, the nanotube yarn bundles are oxidized by chemical treatments to reduce hydrophobicity and improve water absorption and wettability.

In accordance with the disclosure, an exemplary oxidizing agent may be an oxidizing gas or oxidizing solution.

The exemplary strong oxidizing gas may be ozone or chlorine.

The exemplary strong oxidizing solution may be a strong acid or a combination of two or more strong acids, including but not limited to nitric acid, sulfuric acid, perchloric acid, and a mixture of nitric acid and sulfuric acid, sulfuric acid and potassium dichromate, and sulfuric acid and potassium permanganate.

After the treatment, oxidized nanotube yarns or yarn bundles may lose inter-bundle interactions between adjacent yarn bundles due to the nanotube surface modification and the reduced Van der Waals force. This loss benefits the formation and the stability of the nerve regeneration channels within the scaffold lumen and prevents the channel from collapsing due to strong Van der Waals force. It facilitates nutrition and metabolic waste material exchange through the enhanced permeability of channel walls.

The exemplary treated CNT yarn bundles have increased water absorption shown in Table 1.

TABLE 1

| | | | | H₂O |
|---|---|---|---|---|
| | | | | holding |
| | Weight (g) | Weight (g) | Weight | capacity/ |
| No treatment or | prior to | post- | gain | mg CNT |
| treatment type | treatment | treatment | (%) | yarn |
| Silicone Tube | 0.1563 | 0.1563 | 0.00 | N/A |
| Pristine CNT Yarn | 0.1498 | 0.1520 | 1.47 | 10 mg |
| Ozone-treated Yarn | 0.1782 | 0.1859 | 4.32 | 30 mg |
| (300 min) | | | | |
| Acid-treated Yarn | 0.1721 | 0.1836 | 6.68 | 47 mg |
| (300 min) | | | | |

A silicone tube without any CNT yarn serves as a negative control. Three silicone tubes having pristine CNT yarn (untreated), ozone-treated, and acid-treated yarns are measured for weight gains after soaking one terminus overnight in a vertical position in the same water bath. Each silicon tube and the CNT yarns within the same tubes have substantially the same length. The silicone tube of ozone-treated yarns has a 4.32% weight gain; the silicone tube of acid-treated yarns has a 6.68% weight gain. The silicone tube of untreated pristine CNT yarns has a merely 1.27% weight gain. The water-holding capacities are 10 mg for the pristine CNT yarn silicone tube, 30 mg for the ozone-treated CNT yarn silicone tube, and 47 mg for the acid-treated CNT yarn silicone tube.

### Example Nerve Scaffold

As indicated above, some examples of the presently disclosed nerve scaffolds include a plurality of modified yarns and/or yarn bundles fabricated from various materials, including but not limited to synthetic polymer fibers, nylon fibers, carbon nanofibers, carbon nanotube, or boron nitride nanotubes.

In one embodiment, the plurality of nanotube yarn bundles 104 may comprise true twisted multi-ply and single ply nanotube yarns, untwisted multi-ply and single ply nanotube yarns, and false twisted multi-ply and single ply nanotube yarns. The nanotube yarns provide a smooth and nanoporous surface onto which nerve fibers can grow. The nanotube yarns can also provide a scaffold and mechanical support for the regrowing nerve fiber. Together, these nanotube yarn features make the regrowing nerve fibers and the regenerating nerve as a whole less likely to experience tensile or compressive stresses that can cause neuropathic pain or negatively impact nerve fiber growth.

The present disclosure will refer primarily to oxidized carbon nanotube yarn bundles, but it will be appreciated that modified carbon nanotubes and nanotube yarns and BNNT nanotubes and nanotube yarns and bundles share many of the same advantageous features and are included within the generic term “nanotube yarn bundles.”

The nanotube yarn bundles applied herein mainly serve as structural components for overall regeneration scaffolds and individual channel walls for nerve fiber regeneration. Neuronal tissue and nerve fibers can grow (which may also be referred to as “regeneration” or “regrow”) within and along these channels. They also have additional benefits as they are electrically conductive and biocompatible; the latter will be described below. When connecting two severed nerve segments, nanotube yarn bundles can provide conductive paths for sensory and motion signal transmission from one terminus of a severed nerve to another. The presence of an electrical physiological signal conductive path may further improve nerve tissue regrowth and result in enhanced nerve function recovery with added benefits beyond what may generally be expected for a regenerated nerve. In some examples, it is believed that applying nanotube yarn bundles within a nerve scaffold can restore the function depending on a regenerated nerve to the level of the same nerve before the injury. In some other examples, nanotube yarn bundles, and in particular single ply, false twisted carbon nanofiber yarns, yarn bundles, or modified yarn bundles, can provide a topographically nanoporous surface on which nerve fibers can regrow to an extended range and facilitate low tensile stress regeneration. Together, the features presented herein provide an effective solution to the unmet clinical needs of long-gap peripheral nerve injury repair.

One example of a nerve scaffold of the present disclosure is schematically shown in FIGS. 2A and 2B.

In FIG. 2A, the nerve scaffold 100 includes a tube 102 and a plurality of nanotube yarn bundles 104.

FIG. 2B exemplarily represents a portion of a cross-section view of the nerve regeneration scaffold; it does not include tube 102. The benefits of the nerve scaffold 100 include providing gaps or channels between and defined by the adjacent nanotube yarns and yarn bundles of the plurality. The structural arrangement of nanotube yarn bundles 104 provides a plurality of channels, one of which is indicated as feature 108, for nerve fiber or nerve tissue regeneration. A channel has a minimum of three (3) nanotube yarn bundles to form a wall, or more specifically, a virtual wall with gaps between the nanotube yarn bundles. Feature 106 represents one of the gaps in the channel wall. Adjacent nanotube yarn bundles are two nanotube yarn bundles forming a single virtual wall without a third yarn bundle in between, indicated by α1 in FIG. 2B. One of the adjacent nanotube yarn bundles may be a part of two or more walls for other corresponding channels. For a channel comprising at least four nanotube yarn bundles for its virtual wall, a pair of two nanotube yarn bundles separated by at least another nanotube yarn bundle with a maximal distance are defined to be in a diagonal position; this maximal distance is the diagonal distance, indicated as α2 in FIG. 2B.

Gap 106 allows the movement of large molecules and metabolites and branching out of new nerve fiber branches from the new nerve fiber trucks within the channels (not shown).

Channel 108 is configured and dimensioned to encourage nerve growth. For a customized or personalized nerve regeneration scaffold, the channel sizes are selected according to a particular physiological location and needs of a desired implantable scaffold.

The plurality of nanotube yarn bundles 104 (the fabrication of which is described in the U.S. patent application Ser. No. 16/353,608, which is incorporated herein by reference in its entirety) within tube 102 in FIGS. 1 and 2A are shown as being parallel to one another and distributed unevenly within tube 102. It will be appreciated that this is for convenience of illustration only. Rather, nanotube yarns are assembled into the plurality of nanotube yarn bundles 104 so as to occupy a portion of the tube 102 lumen and may be substantially aligned with one another but not necessarily exactly parallel to one another. It is sufficient in many embodiments for this general alignment of nanotube yarn bundles within the plurality of nanotube yarn bundles 104 to define channels and gaps between the nanotube yarn bundles that roughly correspond to a cross-sectional diameter of the nerve fiber. The channel diameter is generally from 5 μm to 10 μm in diameter, and in some examples, from 8 μm to 15 μm or 5 μm to 20 μm. Even in circumstances in which nanotube yarn bundles may cross one another or be misaligned, having spaces that are at least partially continuous along a length of the tube 102 (i.e., greater than 10% or greater than 20% along a length of the tube 102) and within the range of approximately 5 μm to 15 μm in diameter is sufficient to facilitate nerve regeneration in a way that minimizes the tensile forces described above. The distance of the adjacent nanotube yarn bundles can be 2 μm to 8 μm in some examples, 5 μm to 15 μm or 2 μm to 15 μm in other examples.

Tube 102 can perform any of a number of functions. In some examples, tube 102 separates the region in which nerve fibers can regrow from the severed termini. This region is also ideal for neuronal cells or stem cell implantation. Another function of tube 102 is to help protect existing nerve tissue and its termini and the nerve fiber regeneration process from physical damage or other perturbations that may otherwise reduce the growth rate or the continuity of the regrowing nerve. Tube 102 further shields the region and prevents the fast replicating fibroblast in the surrounding tissue from taking over the region intended for the slower nerve regeneration. Tube 102 also defines an interior space in which carbon nanofiber yarns can be disposed of and configured so as to have a density and an arrangement that facilitates nerve fiber regeneration close to the original native structure, as described herein.

Tube 102 can be fabricated from biocompatible and/or bioresorbable materials. Examples of these materials include silicone, which has the added advantage of being compliant rather than rigid, such as plastic material. This compliance facilitates attachment of the ends of tube 102 to severed nerve segments 101A and 101B via suturing or surgical adhesive, e.g., BioGlue®. Other examples of biocompatible materials that can be used for tube 102 include, but are not limited to poly (methyl methacrylate), poly(tetrafluoroethylene), polyethylene, polyglycolide, polycaprolactam, poly (lactic-co-glycolic acid), poly lactic acid, poly (glycerol sebacate), polysialic acid, polyethylene glycol, polyurethane, collagen, chitosan, silk, and alginate, among others. In another set of embodiments, the tube may be made of carbon material, such as carbon nanotubes, in a form of a wrapped sheet or sheets. The nanotubes in the sheets may have a random orientation or aligned end-to-end orientation when drawn from nanotube forests with respect to the planar orientation of the sheet surface. The nanotube sheets-made tube 102 may offer very low mechanical stiffness while excellent durability and a long lifetime. In some embodiments, tube 102 can be made of biological materials, including xenogenic blood vessels. In specific embodiments, tube 102 may comprise nanotube yarn bundles, pristine (unmodified) or modified (oxidized or functionalized), same or different from nanotube yarn bundles inside of the tube lumen.

In some other examples, the length of a nerve regeneration scaffold can be within any of the following ranges: from 1.0 mm to 1.5 mm; from 1.0 mm to 1.0 cm; from 2.0 mm to 5 mm; from 1.0 cm to 2.0 cm; from 1.5 cm to 2 cm; from 2.0 cm to 5 cm; from 3.0 cm to 8.0 cm, or 1.0 cm to 15.0 cm. The inner diameter of a nerve regeneration scaffold is dimensioned so that tube 102 can be disposed over severed ends of a nerve and sutured or otherwise connected thereto, as illustrated in FIG. 1. In some examples, the lumen diameter can be within any of the following ranges: from 0.5 mm to 2.0 mm, 1.0 mm to 4.0 mm; from 1.0 mm to 5.0 mm, or from 3.0 mm to 10.0 mm.

The plurality of nanotube yarn bundles 104 has a length and an outer diameter similar or smaller in size to the length and the inner diameter of tube 102. This size reduction in either length or outer diameter can be 2%, 5.0%, 10.5%, or up to 50.0% or more to ensure the plurality of nanofiber yarns fit inside the tube lumen.

In some embodiments, the interstitial spaces between nanotube yarn bundles may include one or more polymer matrices to support nerve fiber growth. In some examples, the polymer matrices may be one or more of collagen, gelatin, fibrin, neural extracellular matrix (ECM) molecules, ECM proteins, and their analogs. In some other examples, the polymer matrices may comprise one or more of the glycosaminoglycans.

In some embodiments, the nanotube yarn bundles and/or the interstitial spaces of the nanotube yarn bundles may further comprise one or more proteins or growth factors to enhance growth rates and reduce the time for cell division cycles. One or more proteins or growth factors may facilitate the growth or division of one or more specific cell types. In some examples, the protein or the growth factors may be at least one of a vascular endothelial growth factor (VEGF), a nerve growth factor (NGF), a hepatocyte growth factor (HGF), neuregulin 1, glial-derived neurotrophic factor, pleiotrophin, or brain-derived neurotrophic factor (BDGF). In one specific example, the protein and/or growth factors within the interstitial spaces have a slow-release formulation.

In one of the embodiments, the nanotube yarn bundles or the interstitial spaces of the nanotube yarn bundles may further impregnate with at least one inhibitor to prevent less desirable or undesirable adverse reactions or results. Exemplary inhibitors include, but are not limited, foreign body-type multinucleated giant cell inhibitors (FBGC-I).

In another embodiment, FBGC-I may be selected from at least a diacyl-glycerol kinase inhibitor, a protein kinase C inhibitor (PKC), or a combination thereof. Exemplary diacyl-glycerol kinase inhibitors include but are not limited to R59022 and R59949 (3-{2-(4-[bis-(4- fluorophenyl)methylene]-1-piperidinyl)ethyl}-2,3-dihydro-2-thioxo-4(1H)quinazolinone) or their analogs or derivatives. Exemplary PKC inhibitors include but are not limited to (±)-1-(5-Isoquinolinesulphonyl)-2-methylpiperazine dihydrochloride and calphostin C.

In yet another embodiment, the interstitial spaces of the nanotube yarn bundles may include one or more polymer matrices in combination with one or more proteins. In a specific example, the interstitial spaces include collagen in combination with the nerve growth factor.

In some examples, polymer matrices and/or protein growth factors may have a concentration gradient from the proximal end of the nerve regeneration scaffold to the distal end of the scaffold or vice versa.

In other examples, the proper spacing between adjacent nanotube yarn bundles of the plurality of nanotube yarn bundles 104, indicated above, and the number of the channels can be measured indirectly as the percentage of nanotube yarn volume relative to the total volume of nerve scaffold lumen. This percentage can be greater than 0.1%, greater than 2%, smaller than 10%, or smaller than 30%. In some other examples, nerve fiber regrowth fails to emerge when the percentage of nanotube yarn volume relative to the scaffold lumen volume is smaller than 0.1% or greater than 30%. The nerve fiber regrowth may emerge when the nanotube yarn volume percentage falls within the 0.1% to 30% range with sufficient functional recovery detected by electrophysiological measurements in the volume percentage of 2% to 10%.

In some examples, it has been found experimentally that false-twisted nanofiber yarns in the volume percentage range of from 2% to 10% have been found to have inter-fiber space distances between 8 μm and 10 μm.

A nerve regeneration scaffold may include any number of nanotube yarns ranging from 10 to 3,000 or 10 to 8,000.

An exemplary embodiment of the present disclosure incorporates modified nanotube yarn bundles instead of pristine nanotube yarn bundles. The modification includes oxidation by an oxidizing gas, an oxidizing chemical liquid, or a combination thereof. The modified nanotube yarn bundles demonstrate improved wettability measured by water absorption, as described above.

### Study Results of Animal Model

Five animal groups (rats) received implantations of nerve regeneration scaffolds comprising different nanotube yarn bundles. Each animal group in the study included at least five rats in a sciatic nerve transaction model. The five groups are autograft (AG), pristine CNT yarn (P-CNT), oxidized CNT yarn (O-CNT), strong acid-treated CNT yarn (SA-CNT), and silicon tube alone (ST) groups. These rats were tested at eight weeks and sixteen weeks post-surgical implantation to demonstrate the effects of different nanotube yarn bundles in the nerve regeneration scaffold.

Muscle weight loss is a common clinical symptom caused by nerve injury. A gain of muscle weight is an indication of gaining effective functional recovery. Muscle ratio comparing a muscle weight from an injured site vs. that from a healthy contralateral site is often an excellent indicator to demonstrate the difference between various therapies. The effects of different nanotube yarn bundles on the tibialis anterior (TA) and gastrocnemius (GA) muscle ratios are presented in Table 2 below.

TABLE 2

| Group | Week No. | AG | P-CNT | O-CNT | SA-CNT | ST |
|---|---|---|---|---|---|---|
| TA (% ± | 8 Wks | 50 ± 0.01 | 25.4 ± 0.01 | 27.2 ± 0.01 | 28.9 ± 0.02 | 26.5 ± 0.01 |
| SE) | 16 Wks | — | 25 ± 0.03 | 25.6 ± 0.05 | 58.3 ± 0.05 | 19.5 ± 0.01 |
| GA (% ± | 8 Wks | 38.6 ± 0.02 | 21.2 ± 0.00 | 20.9 ± 0.00 | 22.9 ± 0.01 | 18.7 ± 0.01 |
| SE) | 16 Wks | — | 22.6 ± 0.02 | 25.4 ± 0.05 | 48.8 ± 0.04 | 17.8 ± 0.01 |

At eight weeks, the ratios of TA and GA were significantly greater in the AG group compared across all CNT groups. There are weight gains for the O-CNT group over the P-CNT group, while the SA-CNT group demonstrates clear advantages over the P-CNT groups. At sixteen weeks, both TA and GA ratios are more than doubled over the corresponding TA and GA ratios of the P-CNT groups.

The electrophysiological measurements are performed, and the results are presented below in Table 3. At eight weeks and sixteen weeks, standard compound muscle action potentials (CMAP) were measured and recorded after stimulation using the VikingQuest system (Nicolet Biomedical, Madison, WI, the U.S.A.). The CMAP is an electromyography study with the summation of a group of simultaneous action potentials from several muscles covered by the study. It reflects the status of a motor unit. The onset latency and peak-to-peak amplitude of the CMAPs on the experimental side of the animal are recorded.

TABLE 3

| Group | Week No. | AG | P-CNT | O-CNT | SA-CNT | ST |
|---|---|---|---|---|---|---|
| Recovery | 8 Wks | 100 (8/8) | 71.4 (5/7) | 75 (6/8) | 100 (9/9) | 0 (0/9) |
| Rate (%) | 16 Wks | — | 71.4 (5/7) | 100 (5/5) | 100 (6/6) | 0 (0/5) |
| NCV | 8 Wks | 4.61 ± 0.23 | 4.8 ± 0.4 | 4.15 ± 0.19 | 3.97 ± 0.08 | — |
| (ms ± SE) | 16 Wks | — | 5.14 ± 0.3 | 3.28 ± 0.21 | 4.01 ± 0.2 | — |
| Amplitude | 8 Wks | 4791.1 ± | 451.1 ± 168 | 702.7 ± | 728.3 ± | — |
| (μV ± SE) | | 1518.2 | | 400.4 | 157.9 | |
| | 16 Wks | — | 1841.2 ± | 5453.4 ± | 8215.5 ± | — |
| | | | 1038.3 | 2195.2 | 2813.5 | |

At eight weeks, CMAPs were detected in five of seven rats in the P-CNT group (71.4%), six of eight rats in the O-CNT group (75.0%), nine of nine rats in the SA-CNT group (100.0%), and eight of eight rats in the AG group (100.0%). At 16 weeks, the same tests were repeated, with results showing no improvement for the P-CNT group (71.4%) and further improvements for the O-CNT group (100.0%). No CMAPs were observed in the ST group. This data set presents strong evidence to demonstrate the superiority of the modified and oxidized nanofiber yarns in functional recovery.

The mean latency and the mean amplitude of the nerve conduction velocity (NCV) with standard error (SE) at eight weeks were 4.61±0.23 ms and 4,791.1±1,518.2 μV in the AG group; 4.80±0.40 ms and 451.5±168 μV in the P-CNT group; 4.15±0.19 ms and 702.7±400.4 μV in the O-CNT group; and 3.97±0.08 ms and 728.3±157.9 μV in the SA-CNT group.

At 16 weeks, the NCV plus SE results were 5.14±0.3 ms and 1,841.2±1,038.3 μV in the P-CNT group; 3.28±0.21 ms and 5,453.4±2,195.2 μV in the O-CNT group; and 4.01±0.2 ms and 8,215.5±2,813.5 μV in the SA-CNT group.

The NCV and amplitude testing data from both modified nanofiber yarn groups present a continuous recovery significantly surpassing the P-CNT group. The NCV and amplitude testing data from both modified nanofiber yarn groups present a continuous recovery significantly surpassing the P-CNT group. At 8 weeks, the NCV data of the O-CNT and SA-CNT groups demonstrated initial restoration of electrochemical impulse propagation comparable to the P-CNT group, although to a level less than the AG group with respect to the signal amplitudes. The NCV amplitude data collected at 16 weeks strongly suggest the advantages of O-CNT and especially the SA-CNT over the P-CNT. As the CMAP and NCV are often indicative of motor function status, the embodiments of the present disclosure further suggest a preference in motor function recovery over sensory function recovery.

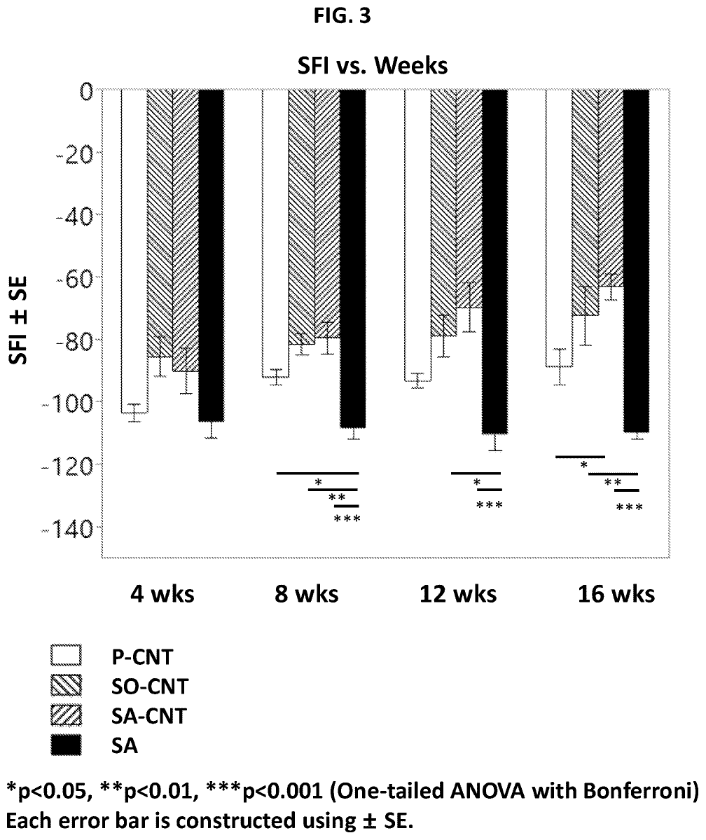

The sciatic functional index (SFI) is a widely used metric for quantitative studies of neurological pathology and potential treatment. This index correlates to nerve fiber/axon diameter ratio and myeline thickness/axon diameter ratio, and a reduction indicates functional recovery. The measurements were taken in a 4-week increment, and the results are shown in FIG. 3.

The indices provided in FIG. 3 show a general improvement across all implanted groups, as demonstrated by the ST group vs. the implanted groups. Among the implanted groups, the SA-CNT group presents the best recovery; the O-CNT group (shown as SO-CNT in FIG. 3) has a better recovery compared to the P-CNT group at the end of Week 8, Week 12, and Week 16.There is a significant difference in the SA-CNT group (−63.2±4.3) compared with the P-CNT group (−88.9±5.7).

Additional histological and immunochemical studies were performed on the longitudinal sections of the harvested nerve tissue from the scaffolds by anti-neurofilament antibody and Schwann cell-specific S100 protein antibody. The staining revealed the presence of axons and myelin sheaths at eight weeks post-implantation. The regrowth extends from the proximal junction of the severed sciatic nerve and scaffold to the distal terminus of the scaffold in all CNT groups. In contrast, no axonal regeneration was observed in the silicon group (data not included). Furthermore, among all CNT groups, the SA-CNT group showed a significant increase in axonal regeneration compared with the P-CNT group, which are apparent based on the immunochemical staining of neurofilaments and Schwann cells at the distal terminus. There was no statistically significant difference in the number of cell nuclei (DAPI) at the proximal and distal regenerated nerve ends between groups (see Table 4 below).

TABLE 4

| Group | Tissue Location | AG | P-CNT | O-CNT | SA-CNT | ST |
|---|---|---|---|---|---|---|
| No. of NF | Proximal | 9741 ± | 9334 ± 794 | 8622 ± 477 | 8914 ± 430 | 8419 ± 347 |
| (±SE) | (no/mm²) | 782 | | | | |
| | Distal | 9016 ± | 6847 ± 696 | 7475 ± 505 | 8269 ± 329 | 1692 ± 316 |
| | (no/mm²) | 803 | | | | |
| | Distal/Proximal | 92.2 ± | 74.1 ± 5.6 | 86.6 ± 2.4 | 93.2 ± 1.5 | 19.5 ± 3.0 |
| | (%) | 3.6 | | | | |
| No. of S10 | Proximal | 9694 ± | 9584 ± 820 | 9122 ± 455 | 9508 ± 474 | 9144 ± 323 |
| (±SE) | (no/mm²) | 934 | | | | |
| | Distal | 9463 ± | 7297 ± 743 | 8092 ± 490 | 8886 ± 406 | 3176 ± 362 |
| | (no/mm²) | 926 | | | | |
| | Distal/Proximal | 97.8 ± | 77.6 ± 6.7 | 88.9 ± 2.3 | 93.9 ± 1.9 | 34.7 ± 4.1 |
| | (%) | 3.0 | | | | |

The analysis results of the CD68 positive cells and the foreign body-type multinucleated giant cells (FBGCs) further support one of the embodiments and the significance of the present disclosure.

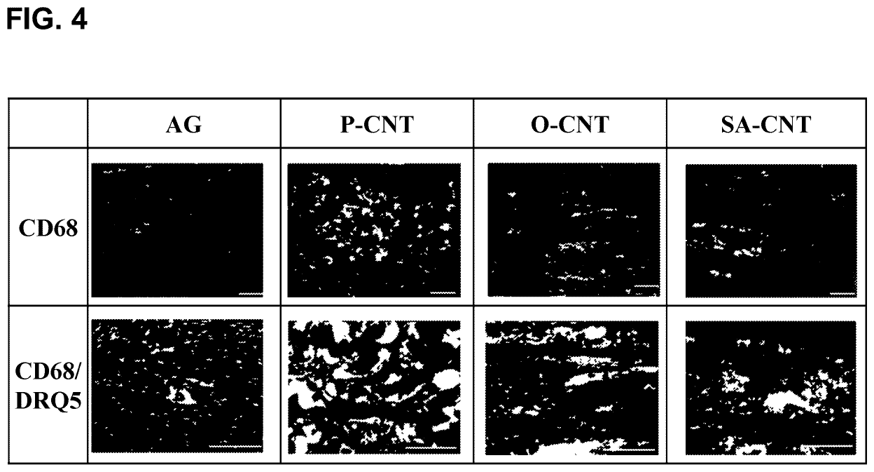

CD68 is a marker for macrophage lineage in circulation and tissue. Staining in the proximal longitudinal section of the regenerated tissue of different treatment groups is presented herein (see FIG. 4). In the P-CNT group, there is significant CD68 staining positive (CD68⁺) cell staining, while CD68⁺ staining is dramatically reduced in the O-CNT group and the SA-CNT group, although the O-CNT group and the SA-CNT group still show a slightly elevated level of CD68⁺ staining.

The FBGCs are the fusion form of macrophages. They are a prominent cell type on implanted biomaterial and have long been regarded as hallmark features of chronic inflammation.

In FIG. 4, DRAQ5, a known nuclei staining agent, demonstrated the formation, increase, or reduction of FBGCs (see clusters of intense signals from DRAQ5 plus CD68⁺ staining images) compared to the CD68⁺ staining images and among several CNT groups. At the proximal longitudinal section of the regenerated tissue, FBGCs are observed around the CNT fibers in all CNT groups. The FBGC numbers are decreased significantly in the O-CNT and SA-CNT groups compared to P-CNT group, with a p-value less than 0.01 (O-CNT compared to P-CNT) or less than 0.001 (SA-CNT compared to P-CNT) (see Table 5 below).

TABLE 5

| Group | AG | P-CNT | O-CNT | SA-CNT |
|---|---|---|---|---|
| FBGC (N/0.1 | 0.3 ± 01 | 15.4 ± 1 | 11.1 ± 0.7 | 10.2 ± 0.8 |
| mm² ± SE) | | | | |

The reduction of both CD68⁺ cells and the foreign body giant cells around the implants after hydrophilizing the nanotube yarn bundles may be construed as a new strategy to solve implantation rejection. Small molecule inhibitors against to FBGC have been suggested, developed, and reported to reduce or eliminate the FBGC formation. Incorporating FBGC inhibitors incorporated into the scaffold, especially a slow-release form, may constitute another embodiment of the present disclosure for further reduction of FBGC. FBGC inhibitors can be selected from one or more of diacylglycerol kinase inhibitors, protein kinase C inhibitors, and their analogs or derivatives,

The present disclosure may further include one or more anti-inflammatory agents in the scaffold. It may include methods for nerve tissue repair and regeneration by applying any embodiments disclosed herein.

### Ex Vivo Neuronal Cell Culture and Use

Various nanofibers and nanotubes with different properties have been explored and researched for ex vivo uses and testing, either for academic research purposes or for pharmacological and toxicological testing services, relying on expanded neuronal cell populations. Functionalized nanotubes and/or nanotube yarn bundles with increased hydrophilicity and reduced undesirable reactions may modulate neuronal growth, such as neurite outgrowth of hippocampal neurons in culture. Ex vivo expansion of neuronal stem cells and glial cells may also depend on the exemplary aspects of the functionalized nanotubes presented herein. The resulting cell populations are ideal for scientific research, drug safety tests, and cell transplantation for injuries or neurodegenerative diseases.

In one embodiment of the present disclosure, modified nanofiber yarns, yarn bundles, or modified nanofiber sheets may be used as substrates for the expansion of harvested primary neuronal tissues and cells, purified primary neuronal cells, or established neuronal cell lines for greater cell population growth when compared to unmodified nanotube material. They may also serve as substrates for stem cell differentiation into neuronal cells, optionally in the presence of selected one or more growth factors. The modified nanofiber yarn bundles may be configured as a scaffold or randomly oriented in ex vivo culture environments. The modified nanofiber sheets may have nanofibers aligned or randomly dispersed in a planar direction of the sheets.

Further embodiments of this disclosure are to apply the nerve regeneration scaffold in an artificial system to grow nerve tissue or apply the modified nanotubes, nanotube yarns or bundles in artificial organs research and production.

### Further Considerations

The foregoing description of the embodiments of the disclosure has been presented for the purpose of illustration; it is not intended to be exhaustive or to limit the claims to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the invention, which is set forth in the following claims.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the present disclosure in its aspects. Although the invention has been described with reference to particular means, materials and embodiments, the invention is not intended to be limited to the particulars disclosed; rather the invention extends to all functionally equivalent structures, methods, and uses such as are within the scope of the appended claims.

The illustrations of the embodiments described herein are intended to provide a general understanding of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A nerve regeneration scaffold comprising:
a tube having a first end and a second end opposite the first end, the tube defining a lumen having a diameter and openings at the first end and the second end, the tube comprising a biocompatible material;
one or more modified nanotube yarn bundles disposed within the lumen and extending from the first end to the second end; and
an interstitial space between the modified nanotube yarn bundles and inside the lumen; wherein
the modified nanotube yarn bundle comprises at least two nanotube yarns;
the modified nanotube yarn bundles define a plurality of channels from the first end to the second end and a plurality of gaps between the modified nanotube yarn bundles; and
the modified nanotube yarn bundles are oxidized bundles of nanofiber yarns.

2. The nerve regeneration scaffold of claim 1, wherein the modified nanotube yarn bundles have a water absorption rate of at least 18 mg per 1 mg of the modified nanotube yarn bundles.

3. The nerve regeneration scaffold of claim 1, wherein the nanotube yarn bundles are false twisted nanofiber yarns.

4. The nerve regeneration scaffold of claim 1, wherein the nanotube yarns are single-ply nanofiber yarns.

5. The nerve regeneration scaffold of claim 1, wherein the nanotube yarns are multi-ply nanotube yarns.

6. The nerve regeneration scaffold of claim 1, wherein a diagonal distance between nanofiber yarn bundles defining one of the channels is from 5 μm to 20 μm.

7. The nerve regeneration scaffold of claim 1, wherein a distance between adjacent nanotube yarn bundles defining one of the channels is from 2 μm to 15 μm.

8. The nerve regeneration scaffold of claim 1, wherein a collective volume of the modified nanotube yarn bundles is from 0.1% to 30% of the lumen volume.

9. The nerve regeneration scaffold of claim 1, wherein at least one of the modified nanotube yarn bundles comprises 10 to 3000 nanotube yarns.

10. The nerve regeneration scaffold of claim 1, wherein diameters of nanotube yarns range from 5 μm to 30 μm.

11. The nerve regeneration scaffold of claim 1, wherein the tube comprises at least a first polymer.

12. The nerve regeneration scaffold of claim 11, wherein the first polymer is a bioabsorbable polymer.

13. The nerve regeneration scaffold of claim 1, wherein the diameter of the lumen ranges from about 1.0 mm to about 21 mm.

14. The nerve regeneration scaffold of claim 1, wherein the nanotube yarns comprise carbon nanotubes.

15. The nerve regeneration scaffold of claim 1, wherein the nanofiber yarns comprise boron nitride nanotubes.

16. The nerve regeneration scaffold of claim 1, wherein the interstitial space between the modified bundles of nanofiber yarns comprises at least a second polymer between the modified nanotube yarn bundles.

17. The nerve regeneration scaffold of claim 16, wherein the second polymer is collagen, gelatin, or a matrix protein.

18. The nerve regeneration scaffold of claim 1 further comprising at least a protein.

19. The nerve regeneration scaffold of claim 18, wherein the protein is a growth factor selected from a vascular endothelial growth factor, a nerve growth factor, a hepatocyte growth factor, neuregulin 1, glial-derived neurotrophic factor, pleiotrophin, a fibrin matrix gel, or a combination of.

20. The nerve regeneration scaffold of claim 1, further comprising at least one foreign body-type multinucleated giant cell inhibitor.

21. The nerve regeneration scaffold of claim 1, wherein the minimum length of the nerve regeneration scaffold is 10 mm.
